(19) United States
(12) Patent Application Publication
(10) Pub. No.: US 2007/0003582 A1
(43) Pub. Date: Jan. 4, 2007

(54) Medicine for the treatment of acne and for reversing the signs of age and sun damage and method for using same

(76) Inventor: Madalene C.Y. Heng, Northridge, CA (US)

Correspondence Address: Sandy Lipkin;Law Offices of Sandy Lipkin, Suite 1180, 300 Esplanade Drive, Oxnard, CA, 93036, US

(21) Appl. No.: 11/517,121

(22) Filed: Sep. 6, 2006

Related U.S. Application Data
(63) Continuation-in-part of application No. 10/723,191, filed on Nov. 25, 2003.

(51) Int. Cl. A61K 36/886; A61K 9/00

(52) U.S. Cl. 424/400; 424/744

(57) ABSTRACT

A chemical compound and a method for its use as a topical medication that can reverse the effects of sun-damage, reverse the signs of aging, demonstrate anti-scarring properties and allow for the treatment of acne while still permitting the patient the ability to simultaneously expose the affected area to the sun without damage that includes a combination of curcumin, water, alcohol, cellulose, anti-inflammatory agent, carbomer, diazolinyl urea, triethanolamine, EDTA and an acidic component that maintains the optimal pH of between 4.5 and 5.5

### REFERENCE TO PRIOR APPLICATION

This application is a continuation-in-part of application Ser. No. 10/723,191, filed Nov. 25, 2003, entitled A MEDICINE FOR TREATING ACNE by the present inventor.

### BACKGROUND OF THE INVENTION

1. Field of the Invention

The present invention relates generally to the field of dermatology, and particularly to a chemical compound and a method for its use as a topical medication that can reverse the effects of sun-damage, reverse the signs of aging, demonstrate anti-scarring properties and allow for the treatment of acne while still permitting the patient the ability to simultaneously expose the affected area to the sun without damage.

2. Description of the Prior Art

Acne is a pleomorphic skin disease, commonly referred to as acne vulgaris. Acne is characterized by blackheads, whiteheads, papules, pustules and various size nodules and scars. The disease involves the hair follicle and is characterized by the enlargement and infection of the sebaceous glands and ducts. A sebaceous gland is a gland which draws into the hair follicle and produces and liberates sebum, which is a mixture composed of fat, cellular debris and keratin. A normal hair follicle is lined by protective layers of cells, which is known as the stratum corneum.

When the sebaceous gland is located in association with a hair follicle, it forms a thickened out-pushing from the side of the developing follicle near the epidermis. Central cells in these sebaceous glands form oil droplets within the cytoplasm. These cells disintegrate to liberate the sebaceous oil known as sebum. In healthy skin, the central passage through which a hair shaft passes is to remain open and unobstructed to allow the sebum secreted by the sebaceous glands to easily reach the surface of the skin.

Sebum is a complex liquid liberated by the breakdown of the sebaceous cells and is intimately associated with the development of acne. A perifolliculitis results following rupture of the follicular contents (sebum) into the dermis. This occurs secondary to obstruction of the opening of the sebaceous follicle by a whitehead or a blackhead. The inflammatory response eventually heals, but generally with a scar formation.

Acne is an extremely common occurrence in conjunction with young adults. When the hair follicle becomes plugged and the sebum is not able to be released, bacteria causes an inflammation to occur which frequently results in a reddish pimple-like appearance on the surface of the skin. Plugging of the hair follicle by excessive layers of stratum corneum cells is the underlying abnormality leading to the development of acne lesions.

The most common treatment for acne is he topical application of benzoyl peroxide. The purpose of the benzoyl peroxide is to kill the bacteria that accumulates in the clogged follicles. It has been found when using benzoyl peroxide that the approximate percent decrease in plugged follicles ranges between 15 and 22 percent in most cases. Therefore, benzoyl peroxide is not effective in unplugging the follicles. Its main efficacy in the treatment of acne is its ability to decrease inflammatory pimples by killing bacteria.

Acne is an inflammatory skin disease caused by the plugging of the orifices of the hair follicles, i.e., pores, by skin scales. The plugged follicle caused by acne is often referred to as a comedone. The plugging results in the damming back of the sebaceous secretion from the oil, i.e., sebaceous oil, which enlarge and then colonize with yeast cells known as Pityosporum ovale and bacteria known as Proprionobacterium acnes or P. acnes. These yeasts and bacteria can cause an inflammatory reaction, resulting in acneform abscesses or pimples. By unplugging the pores with agents that remove the comedone, i.e., comedolytic agents, the acne condition improves.

Retinoic acid gel is an effective comedolytic agent. Retinoic acid gel is currently used to treat acne. However, retinoic acid gel is irritating, and makes the skin red and scaly. It also makes the patient sensitive to the sun, and therefore is only used at night. It has no effect on reducing the pigmentation and the scarring from acne abscesses.

Curcumin gel is a known phosphorylase kinase inhibitor and the current inventor owns a patent for the use of phosphorylase kinase inhibitors in leading to a resolution of psoriasis. (U.S. Pat. No. 5,925,376 to Heng). The resultant effect of blocking phosphorylase kinase activity in injured skin results in both anti-proliferative (anti-scaling) properties as well as anti-inflammatory properties.

It has been shown that phosphorylase kinase activity stimulates the breakdown of glycogen by phosphorylation in order to generate energy supplies in the form of ATP (adenosine tri-phosphate) for use by inflammatory cells and other skin cells in psoriatic epidermis.

Curcumin is listed as one of many possible ingredients in U.S. Pat. No. 6,294,186 to Beerse et al. for anti-micorobial compositions comprising a benzoic acid analog and a metal salt. The Beerse patent covers an invention that only peripherally mentions the use of curcumin as part of an anti-microbial composition and peripherally mentions that compositions mentioned in that patent could conceivably treat acne. However, there is no mention of the anti-scarring, anti-aging and reversal of sun damage effects discovered by the composition containing curcumin covered in the instant invention, and a specific composition for the treatment of acne is not disclosed.

Accordingly, the composition described herein is a novel improvement over the prior art that yields unexpected results, including the treatment of acne, anti-scarring properties, reversal of skin damage, reduction in the effects of aging as well as the ability of the patient to use the treatment while simultaneous exposing the subject skin to the sun.

### SUMMARY OF THE INVENTION

The preferred embodiment of the present invention teaches a composition for a medicine that reverses the undesirable effects of skin injury by inhibiting phosphorylase kinase, resulting in the healing of damaged skin, including acne, burns and sun damage with minimal pigmentation and scarring while allowing simultaneous exposure to the sun comprising: curcumin; water; alcohol; cellulose; anti-inflammatory agent; carbomer; diazolinyl urea; triethanolamine; acidic component; and ethylendiaminetetraacetic acid.

A second embodiment involves a method of preparing a medicine that reverses the undesirable effects of injury by inhibiting phosphorylase kinase, resulting in the healing of damaged skin, including acne, burns and sun damage with minimal pigmentation and scarring while allowing simultaneous exposure to the sun comprising the steps of: thoroughly mixing a certain amount of water, alcohol, cellulose, anti-inflammatory agent, diazolinyl urea, triethylendiaminetetraacetic acid, and carbomer; adding a certain amount of curcumin; reading a pH measurement of the resulting mixture; and adding a sufficient amount of an acid to create a pH in the range of 2-10.

A third embodiment involves a method for treating skin that reverses the undesirable effects of injury by inhibiting phosphorylase kinase, resulting in the healing of damaged skin, including acne, burns and sun damage with minimal pigmentation and scarring while allowing simultaneous exposure to the sun comprising: application of a compound to a patient's skin, said compound further comprising: curcumin; water; alcohol; cellulose; anti-inflammatory agent; carbomer; diazolinyl urea; triethanolamine; acidic component; and ethylendiaminetetraacetic acid.

All three of the above embodiments can be further modified by the addition of a sufficient amount of acidic agent to lower the pH of the composition to range between 2 and 10.

All three of the above embodiments can be further modified by further defining the acidic component is an acid from the following the group: citric acid, ascorbic acid, azelaic acid, glycolic acid, acetic acid, hydroxy acid.

All three of the above embodiments can be further modified by further defining the alcohol is an alcohol from the following group: isopropyl alcohol, ethyl alcohol or any other alkyl alcohol.

All three of the above embodiments can be further modified by further defining the anti-inflammatory agent as aloe vera or a polyphenol from green tea or red wine.

All three of the above embodiments can be further modified by further defining the chemical composition as being between 60-85% by volume water, between 5-30% alcohol by volume, between 0.5-10% by volume cellulose, between 0.1-5% by volume urea, between 0.5-5% by volume carbomer and between 0.0001-10% by weight curcumin.

It is the primary object of the present invention to provide a chemical composition that can be used to unplug the clogged skin pores that cause acne.

It is yet another object of the invention to provide a chemical composition that reverses the undesirable effects of injury by inhibiting phosphorylase kinase, resulting in the healing of damaged skin, including acne, burns and sun-damaged skin with minimal pigmentation and scarring.

### DETAILED DESCRIPTION OF A PREFERRED EMBODIMENT

Curcumin is the active ingredient in the instant mixture. In its pure form, curcumin is not absorbed through the skin. The instant invention provides a formulation that allows for absorption of curcumin through the skin with efficacy.

Curcumin is a natural inhibitor of the enzyme, phosphorylase kinase (PhK). PhK is involved in providing energy in the form of ATP (adenosine tri-phosphate) from the breakdown of glycogen to energize many cellular processes. Frequently, these inflammatory pathways lead to unsightly skin appearance, such as redness, scaling, pigmentation and scarring. By inhibiting PhK activity with curcumin gel, these unsightly skin changes are improved.

The instant invention involves the application of a composition that is a combination of curcumin, alcohol (isopropyl alcohol can be used, but ethyl alcohol or another alcohol can also be used), an anti-inflammatory agent such as aloe vera, cellulose, carbomer, diazolinyl urea, triethanolamine, EDTA, acid (such as citric acid, ascorbic acid, azelaic acid, glycolic acid, acetic acid or hydroxy acids) and water. The alcoholic gel that is formed by this mixture maintains the “dried out state” of the stratum corneum, thereby keeping the stratum corneum compact.

The gel type composition of the present invention can be formed containing different quantities of different ingredients. However, all composition will include an acidic component and also an agent that stimulates the activity of an enzyme which digests the cementing substance contained within the stratum corneum cells of the epidermis thereby unplugging the follicles. The acidic component is from the group consisting of citric acid, ascorbic acid, azelaic acid, glycolic acid, acetic acid and hydroxy acids. The gel is formulated by combining a certain amount of water, alcohol, cellulose, anti-inflammatory agent such as aloe vera, diazolinyl urea, triethylendiaminetetraacetic acid and carbomer together and thoroughly mixing same.

Curcumin is then added and also is thoroughly mixed. The pH of this substance is then measured and recorded. Generally, the pH will be around 7. The final agent, which is one of the acids with generally citric acid being preferred, is then to be added until the overall pH of the gel is within the range of 4.5 to 5.5 with 5 being preferred. A synergistic response in stimulating the enzyme to unplug the follicle can also be achieved by combining high doses orally of Vitamin A administered daily in the dosage amount of 150,000 I.U. to 500,000 I.U. (preferably 300,000 I.U.).

A typical composition for the gel of the present invention would be: between 60-85% by volume of water, between 5-30% by volume alcohol, between 0.5 to 10% by volume cellulose, 0.1 to 5% by volume urea and between 0.5 to 5% by volume carbomer. The amount of curcumin is 0.0001 to 10% by volume. The amount of citric acid is dependent upon the required level to achieve a pH approximating 5.

When used as a first layer on the skin, the instant composition protects the follicles from the moisturizing effects of topical preparations such as creams, make-up, sunscreens, moisturizers and the like, when applied on top as a second layer. This is the only way the acne individual is able to use sunscreens and makeup without plugging up their pores.

In addition, using the correct pH in the composition, i.e., a pH of around 5, the instant composition has been observed to possess comedolytic properties, i.e., it unplugs follicles by removing the comedone.

The comedone consists of multiple layers, from hundreds to thousands, of stratum corneum, thus encroaching on the patency of the hair follicle through which the sebum from the oil glands normally drain to the skin surface. The layers of the stratum corneum are normally “glued together” by an intercellular substance called “desmoglein,” which serves as a “glue” between the layers of stratum corneum. If the desmoglein or its attachments to the stratum corneum is disrupted, the layers of stratum corneum detach from one another. It has been shown experimentally that one can cause the layers of the stratum corneum to break apart by changing the pH of the composition. This is believed to be achieved through the activation of a protease enzyme which is activated at around a pH of 5. When activated, the protease digests either desmoglein or its attachments to the stratum corneum cells, thereby causing the individual stratum corneum cells to separate from one another and detach.

This protease may originate from inflammatory cells such as neutrophils or even from bacteria such as Proprionibacterium acnes or lipophilic yeasts such as Pityosproum ovale colonizing the hair follicles. In normal follicles, the acne bacteria releases enzymes which convert the stratum corneum cell membrane triglycerides (pH 7) to free fatty acids (pH 5), thereby activating another protease, which is believed to be either human or microbial, which cause the stratum corneum layers to detach.

The above mechanism may be a survival feature for these bacteria which is needed in order for the bacteria to maintain the patency of the follicular orifices. When the stratum corneum is swollen by the use of moisturizers, the available space occupied by the bacteria is compromised, as is their ability to acidify the follicles. The alcoholic gel composition of this curcumin gel preparation keeps the stratum corneum compact, thus allowing for maximal patency of the hair follicular openings.

The illustrations and examples provided herein are for explanatory purposes and are not intended to limit the scope of the appended claims. This disclosure is to be considered an exemplification of the principles of the invention and is not intended to limit the spirit and scope of the invention and/or claims of the embodiment illustrated. Those skilled in the art will make modifications to the invention for particular applications of the invention.

What is claimed is:

1. A composition for a medicine that reverses the undesirable effects of skin injury by inhibiting phosphorylase kinase, resulting in the healing of damaged skin, including acne, burns and sun damage with minimal pigmentation and scarring while allowing simultaneous exposure to the sun comprising:
curcumin;
water;
alcohol;
cellulose;
anti-inflammatory agent;
carbomer;
diazolinyl urea;
triethanolamine;
acidic component; and
ethylendiaminetetraacetic acid.

2. A composition according to claim 1 wherein the amount of said acidic agent added is sufficient to lower the pH of the composition to range between 2 and 10.

3. A composition according to claim 1 wherein said acidic component is an acid from the following the group: citric acid, ascorbic acid, azelaic acid, glycolic acid, acetic acid, hydroxy acid.

4. A composition according to claim 1 wherein said alcohol is an alcohol from the following group: isopropyl alcohol, ethyl alcohol or any other alkyl alcohol.

5. A composition according to claim 1 wherein said anti-inflammatory agent is aloe vera.

6. A composition according to claim 1 wherein said composition contains between 60-80% by volume water.

7. A composition according to claim 1 wherein said composition contains between 5-30% by volume alcohol.

8. A composition according to claim 1 wherein said composition contains between 0.5-10% by volume cellulose.

9. A composition according to claim 1 wherein said composition contains between 0.1-5% by volume urea.

10. A composition according to claim 1 wherein said composition contains between 0.5-5% by volume carbomer.

11. A composition according to claim 1 wherein said composition contains between 0.0001-10% by volume curcumin.

12. A method of preparing a medicine that reverses the undesirable effects of injury by inhibiting phosphorylase kinase, resulting in the healing of damaged skin, including acne, burns and sun damage with minimal pigmentation and scarring while allowing simultaneous exposure to the sun comprising the steps of:
thoroughly mixing a certain amount of water, alcohol, cellulose, anti-inflammatory agent, diazolinyl urea, triethylendiaminetetraacetic acid, and carbomer;
adding a certain amount of curcumin;
reading a pH measurement of the resulting mixture; and
adding a sufficient amount of an acid to create a pH in the range of 2-10.

13. A method according to claim 12 wherein said alcohol is an alcohol from the following group: isopropyl alcohol, ethyl alcohol or any other alkyl alcohol.

14. A method according to claim 12 wherein said acid agent is an acid from the following the group: citric, ascorbic acid, azelaic acid, glycolic acid, acetic acid, hydroxy acid.

15. A method according to claim 12 wherein said anti-inflammatory agent is aloe vera.

16. A method according to claim 12 wherein said composition contains between 60-80% by volume water.

17. A method according to claim 12 wherein said composition contains between 5-30% by volume alcohol.

18. A method according to claim 12 wherein said composition contains between 0.5-10% by volume cellulose.

19. A method according to claim 12 wherein said composition contains between 0.1-5% by volume urea.

20. A method according to claim 12 wherein said composition contains between 0.5-5% by volume carbomer.

21. A method according to claim 12 wherein said composition contains between 0.0001-10% by volume curcumin.

22. A method for treating skin that reverses the undesirable effects of injury by inhibiting phosphorylase kinase, resulting in the healing of damaged skin, including acne, burns and sun damage with minimal pigmentation and scarring while allowing simultaneous exposure to the sun comprising:
application of a compound to a patient's skin, said compound further comprising:
curcumin;
water;
alcohol;
cellulose;
anti-inflammatory agent;
carbomer;
diazolinyl urea;
triethanolamine;
acidic component; and
ethylendiaminetetraacetic acid.

23. A method according to claim 22 wherein the amount of said acidic agent added is sufficient to lower the pH of the composition to range between 2 and 10.

24. A method according to claim 22 wherein said acidic component is an acid from the following the group: citric acid, ascorbic acid, azelaic acid, glycolic acid, acetic acid, hydroxy acid.

25. A method according to claim 22 wherein said alcohol is an alcohol from the following group: isopropyl alcohol, ethyl alcohol or any other alkyl alcohol.

26. A method according to claim 22 wherein said composition is applied to a patient's skin in a concentration of between 0.0001-10% by weight.

27. A method according to claim 22 wherein said anti-inflammatory agent is aloe vera.

28. A method according to claim 22 wherein said composition contains between 60-80% by volume water.

29. A method according to claim 22 wherein said composition contains between 5-30% by volume alcohol.

30. A method according to claim 22 wherein said composition contains between 0.5-10% by volume cellulose.

31. A method according to claim 22 wherein said composition contains between 0.1-5% by volume urea.

32. A method according to claim 22 wherein said composition contains between 0.5-5% by volume carbomer.

33. A method according to claim 22 wherein said composition contains between 0.0001-10% by volume curcumin.
